(19) United States
(12) Patent Application Publication
(10) Pub. No.: US 2020/0196875 A1
(43) Pub. Date: Jun. 25, 2020

(54) METHOD, MODULE AND SYSTEM FOR ANALYSIS OF PHYSIOLOGICAL SIGNALS

(71) Applicants: Adaptive, Intelligent and Dynamic Brain Corporation (AidBrain), Taoyuan (TW); NORDEN E. HUANG, Bethesda, MD (US)

(72) Inventor: NORDEN E. HUANG, Bethesda, MD (US)

(21) Appl. No.: 16/612,405

(22) PCT Filed: May 22, 2018
(86) PCT No.: PCT/US2018/033768

§ 371 Date: Nov. 11, 2019

Related U.S. Application Data
(60) Provisional application No. 62/509,199, filed on May 22, 2017.

(51) Int. Cl. A61B 5/0205; A61B 5/00; A61B 5/04; A61B 5/087; A61B 5/044; A61B 5/0452; A61B 5/026; A61B 5/103; A61B 5/021

(52) U.S. Cl. CPC A61B 5/02055; A61B 5/742; A61B 5/04012; A61B 5/087; A61B 5/021; A61B 5/0452; A61B 5/026; A61B 5/103; A61B 5/7253; A61B 5/044

(57) ABSTRACT

The present disclosure provides a system for analyzing physiological signals. The system comprises a visual output module for rendering a visual output space according to a plurality of analyzed data sets generated by a analysis module, and displaying a visual output, wherein the visual output comprises a first axis representing frequency modulation (FM), a second axis representing amplitude modulation (AM), and a plurality of visual element defined by the first axis and the second axis, and each of the visual elements comprises an accumulated signal strength and the analyzed data sets.

### CROSS-REFERENCE TO RELATED APPLICATION

The present disclosure claims priority to U.S. provisional patent application No. 62/509,199, filed on May 22, 2017, the entirety of which is incorporated herein by reference.

### FIELD

The present disclosure is generally related to analysis of physiological signals. More particularly, the present disclosure is related to analysis of electrical activities of the heart and blood pressure.

### BACKGROUND

Physiological signals provide valuable information for evaluation, diagnosis, or even prediction of physical conditions of a living organism. Each type of physiological signals obtained from a living organism represents the status of a particular system of the living organism.

Various physiological signals can be obtained from a living organism, including but not limited to: electrocardiogram (EKG) signals, electromyogram (EMG) signals, electroretinography (ERG) signals, blood pressure, pulse oximetry (SpO₂) signals, body temperature, and spirometry signals. A plurality of metrics can be obtained from measurement of one or more physiological signals, including but not limited to: electric current, electric impedance, pressure, flow rate, temperature, vibration, breath rate, weight, pulse amplitude, pulse wave velocity, or frequency of physiological events. Also, the metrics can be recorded in a time varying fashion. Metrics can be measured by one or more devices and then stored as the physiological signals. The physiological signals can be further processed into quantitative or qualitative information that are important in clinical evaluation, diagnosis, staging or prognosis.

Physiological signals may be presented by a graph with signal strength or power over time, such as EKG or EMG. However, in frequencies or wave characteristics shown in the graph, noise or disturbances are considered as irrelevant information when conducting analysis of acquired metrics. Moreover, wave patterns hidden in the acquired metrics could be a reference for clinical evaluation, diagnosis, staging or prognosis. Thus, signal processing is a vital part for visualizing and extracting useful information from physiological measurements.

The non-stationary and non-linear nature of many physiological wave signals pose significant obstacles for signal processing. Conventional approaches for signal processing of physiological wave signals have failed to provide an effective solution to the obstacles. For instance, Fourier transformation are often used to interpret linear and stationary wave signals, such as spectrum analysis; however, due to its mathematical nature and probability distribution, Fourier transformation is unable to provide meaningful visualization results from non-stationary and non-linear wave signals.

The Holo-Hilbert spectral analysis (HOSA) is a tool for visualizing non-stationary and non-linear waves. The mathematics behind HOSA has been summarized in Huang et al (Huang, N. E., Hu, K., Yang, A. C., Chang, H. C., Jia, D., Liang, W. K., Yeh, J. R., Kao, C. L., Juan, C. H., Peng, C. K. and Meijer, J. H. (2016). On Holo-Hilbert spectral analysis: a full informational spectral representation for nonlinear and non-stationary data. Phil. Trans. R. Soc. A, 374(2065)). HOSA adopts some of the mathematical methodologies of Hilbert-Huang transformation when analyzing non-stationary and non-linear waves. However, the application of HOSA on analysis of physiological signals has never been explored and exploited.

Due to the lack of adequate signal processing tools, data associated with acquired physiological signals often need to be analyzed by trained professionals, in addition to available algorithms or software embedded instruments. Physiological measurement data could be massive in terms of their quantity and complexity. For instance, a Holter monitor can generate EKG data of an individual continuously for 24 hours. The complexity and amount of the acquired 24-hour EKG data are overwhelming even for well-trained professionals, therefore increasing the chances of missed detection or misinterpretation of EKG deviation or abnormal EKG signals.

Given the non-linear and non-stationary nature of physiological signals and the inherent complexity and quantity of physiological measurement data, there is a need for an efficient and intuitive mean for analysis and visualization of physiological signals.

### BRIEF SUMMARY OF THE DISCLOSURE

It is an object of the present disclosure to provide HOSA-based methods and systems for analysis of physiological signals with wave characteristics.

It is an object of the present disclosure to provide one or more visual outputs of physiological signals.

It is also an object of the present disclosure to provide methods or systems for presenting one or more amplitude-versus-time graphs of EKG signals and blood pressure.

It is also an object of the present disclosure to provide one or more visual outputs of abnormal EKG signals and blood pressure.

It is also an object of the present disclosure to provide applications of HOSA in diagnosis of cardiovascular disorders.

An embodiment of the present disclosure provides a non-transitory computer program product embodied in a computer-readable medium which, when executed by one or more analysis modules, provides a visual output for presenting physiological signals. The non-transitory computer program product comprises a first axis representing frequency modulation (FM), a second axis representing amplitude modulation (AM), and a plurality of visual elements. Each of the visual elements being defined by the first axis and the second axis, and each of the visual elements comprises an accumulated signal strength and a plurality of analyzed data units from a time period. Each of the analyzed data units comprises a first coordinate, a second coordinate, and a signal strength value, the first coordinate is an argument of a FM function from a transformation on a primary intrinsic mode function (IMF), the second coordinate is an argument of an AM function from a transformation on a secondary IMF. Each of the primary IMF is generated from an empirical mode decomposition (EMD) of a plurality of the physiological signals, and each of the secondary IMF is generated from an EMD of the primary IMF, and the accumulated signal strength is an integral of the signal strength value of each of the analyzed data units.

In a preferred embodiment, the first axis is a logarithmic scale of FM, the second axis is a logarithmic scale of AM, the first coordinate is a logarithmic value of the argument of the FM function, and the second coordinate is a logarithmic value of the argument of the AM function.

In a preferred embodiment, the physiological signals are electrocardiogram (EKG) signals, electromyogram (EMG) signals, electroretinography (ERG) signals, blood pressure, oximetry (SpO₂) signals, body temperature, or spirometry signals.

In a preferred embodiment, the signal strength is the amplitude of electric current, electric impedance, pressure, flow rate, temperature, vibration, breath rate, weight, pulse amplitude, pulse wave velocity, or frequency of physiological events within the time period.

In a preferred embodiment, the accumulated signal strength in each of the visual elements is indicated by different colors, grayscales, dot densities, contour lines, or screentones.

In a preferred embodiment, the non-transitory computer program product further comprises one or more contrast lines surrounding the visual elements of one or more of the accumulated signal strengths.

In a preferred embodiment, each of the visual elements comprising a probability for quantifying the statistical significance between at least two other visual outputs.

In a preferred embodiment, the probability for quantifying the statistical significance is a P-value.

In a preferred embodiment, each of the visual elements comprising an area-under-curve (AUC) between at least two other visual outputs.

An embodiment of the present disclosure provides a system for analyzing physiological signals. The system comprises a detection module for detecting the physiological signals, a transmission module for receiving the physiological signals from the detection module and delivering the physiological signals from the detection module and delivering the physiological signals to the analysis module, an analysis module for generating a plurality of analyzed data sets from the physiological signals, and a visual output module for rendering a visual output space according to the analyzed data sets generated by the analysis module, and displaying a visual output. The visual output comprises a first axis representing FM, a second axis representing AM, and a plurality of visual elements defined by the first axis and the second axis. Each of the visual elements comprises an accumulated signal strength and the analyzed data sets, and each of the analyzed units comprises a first coordinate, a second coordinate, and a signal strength value. The first coordinate is an argument of a FM function, and the accumulated signal strength is an integral of the signal strength value of each of the analyzed data units.

In a preferred embodiment, the system further comprises a non-transitory computer program product for presenting physiological signals. The non-transitory computer program product comprises sets of instructions that, when executed by the analysis module, causes the analysis module to perform actions comprising: 1) performing empirical model decomposition (EMD) on the physiological signals to generate a set of primary intrinsic mode functions (IMFs); 2) performing the EMD on the set of primary IMFs to generate a set of secondary IMFs; 3) performing transformations on the set of primary IMFs to generate FM functions; 4) performing transformations on the set of secondary IMFs to generate AM functions; and 5) combining the AM functions and the FM functions to generate a plurality of the analyzed data sets.

In a preferred embodiment, the system comprises an analysis module for generating a set of probabilities for quantifying statistical significance between at least two other visual outputs, and a visual output module for rendering a visual output space according to the set of probabilities, and displaying a visual output. The visual output comprises a first axis of a logarithmic scale of FM, a second axis of a logarithmic scale of AM, and a plurality of visual elements defined by the first axis and the second axis. Each of the visual element comprises a probability for quantifying statistical significance.

In a preferred embodiment, the system comprises an analysis module for generating a set of area-under-curves (AUCs) between at least two visual elements, and a visual module for rendering a visual output space according to the set of AUCs, and displaying an AUC visual output. The AUC visual output comprises a first axis of a logarithmic scale of FM, a second axis of logarithmic scale of AM, and a plurality of AUC visual elements defined by the first axis and the second axis, and each of the AUC visual element comprises an AUC.

An embodiment of the present disclosure provides a non-transitory computer program product embodied in a computer-readable medium which, when executed by one or more analysis module, provides a visual output for presenting physiological signals. The non-transitory computer program product comprises a first axis representing variations of signal strength of the physiological signals within a time period, and a second axis representing signal strengths of the physiological signals. Zero is on the midpoint of the first axis and a threshold value is on a midpoint of the second axis, and the visual output is divided into four quadrants by the threshold value on the second axis and zero on the first axis.

In a preferred embodiment, the physiological signals are transformed into one or more IMFs by EMD. The first axis is a scale of arguments of the variations of the IMFs, and the second axis is the signal strength of the IMFs.

In a preferred embodiment, the IMFs are logarithmized. The first axis is a logarithmic scale of the arguments of the variations of the IMFs. The second axis is a logarithmic scale of the signal strength of the IMFs. A logarithmic value of the threshold value is on a midpoint of the first axis.

An embodiment of the present disclosure provides a system for analyzing physiological signals. The system comprises a detection module for detecting the physiological signals, a transmission module for receiving the physiological signals from the detection module and delivering the physiological signals to the analysis module, an analysis module for generating a primary analyzed data set, and a non-transitory computer program product for presenting physiological signals. The non-transitory computer program product comprises sets of instructions that, when executed by the analysis module, causes the analysis module to perform actions comprising: 1) calculating variations of signal strengths of the physiological signals in a time period; and 2) combining the variations of the signal strengths and the signal strengths of the physiological signals to generate a primary analyzed data set. The program further comprises a visual output module for rendering a visual output space according to the primary analyzed data set from the analysis module, and displaying a visual output comprising a first axis representing the variations of the signal strengths and a second axis representing the signal strengths. Zero is on a midpoint of the first axis and a threshold value is on a midpoint of the second axis, and visual output is divided into four quadrants by the threshold values on the second axis and zero on the first axis.

In a preferred embodiment, the actions performed by the analysis module further comprises: 3) performing EMD on the physiological signals generate one or more IMFs; 4) calculating variations of IMFs in the time period; 5) combining the variations of the IMFs and the primary IMFs to generate a plurality of secondary analyzed data sets.

In a preferred embodiment, the visual output module further comprising rendering another visual output space according to the secondary analyzed data sets from the analysis module, and displaying another visual output comprising a first axis representing a scale of arguments of the variations of the IMFs and a second axis representing a signal strength of the IMFs. Zero is on the midpoint of the first axis and another threshold is on the midpoint of the second axis, and another visual output is divided into four quadrants by another threshold value on the second axis and zero on the first axis.

In a preferred embodiment, the IMFs are logarithmized, the first axis is a logarithmic scale of arguments of the variations of the IMFs, the second axis is a logarithmic scale of the signal strength of the IMFs, and a logarithmic value of the another threshold value is on the midpoint of the first axis.

An embodiment of the present disclosure provides a method for presenting physiological signals. The method comprises: 1) detecting the physiological signals; 2) performing EMD on the physiological signals to generate a set of primary IMFs; 3) performing transformations on the set of primary IMFs to generate FM functions; 4) performing transformations on the set of secondary IMFs to generate AM functions; 5) combining the AM functions and the FM functions to generate a plurality of the analyzed data sets; and 6) rendering a visual output space according to the analyzed data sets.

In a preferred embodiment, the method further comprises 6) logarithmizing the analyzed data sets.

### BRIEF DESCRIPTION OF THE DRAWINGS

Implementations of the present technology will now be described, by way of examples only, with reference to the attached figures.

FIG. 1 is a schematic diagram of a system for analyzing physiological signals in accordance with an embodiment of the present disclosure.

FIG. 2 is a flow diagram of a method for analyzing physiological signals in accordance with an embodiment of the present disclosure.

FIG. 3 is a flow diagram of a method for analyzing electrocardiogram (EKG) signals in accordance with an embodiment of the present disclosure.

FIG. 4 is a flow diagram of a method for analyzing blood pressure in accordance with an embodiment of the present disclosure.

FIG. 5A is a flow diagram of transforming detected signals into a set of primary intrinsic mode functions (IMFs) in accordance with an embodiment of the present disclosure.

FIG. 5B is a flow diagram of an interpolation processes in accordance with an embodiment of the present disclosure.

FIG. 5C is a flow diagram of processes of empirical mode decomposition (EMD) in accordance with an embodiment of the present disclosure.

FIG. 5D is a flow diagram of secondary IMFs generated from envelope functions in accordance with an embodiment of the present disclosure.

FIG. 5E is a flow diagram of transforming primary IMFs into frequency modulation (FM) functions in accordance with an embodiment of the present disclosure.

FIG. 5F is a flow diagram of transforming secondary IMFs into amplitude modulation (AM) functions in accordance with an embodiment of the present disclosure.

FIG. 5G is a schematic diagram of an analyzed data unit in accordance with an embodiment of the present disclosure.

FIG. 6 is a schematic illustration of a visual output of a plurality of analyzed data sets in accordance with embodiments of the present disclosure.

FIG. 7A is a marked-up amplitude-versus-time graph for detected signals in accordance with an embodiment of the present disclosure.

FIGS. 7B, 7C and 7D are IMF modulated signal graphs in accordance with some embodiments of the present disclosure.

FIG. 8 is a plot graph for a plurality of analyzed data sets in accordance with an embodiment of the present disclosure.

FIG. 9 is a marked-up heat map transformed from the plot graph of FIG. 8 in accordance with an embodiment of the present disclosure.

FIG. 10A and FIG. 10B are marked-up visual outputs of the analyzed data sets in accordance with embodiments of the present disclosure.

FIG. 11 is a marked-up visual output with enhanced contrast of the analyzed data sets in accordance with embodiments of the present disclosure.

FIG. 12 is a time-varying graph of blood pressure of a subject in accordance with an embodiment of the present disclosure.

FIG. 13 is an IMF modulated signal graph of blood pressure in accordance with an embodiment of the present disclosure.

FIGS. 14A and 14D are trajectory graphs of blood pressure variation among different time periods of a subject in accordance with embodiments of the present disclosure.

FIGS. 14B and 14C are marked-up trajectory graphs of blood pressure variation among different time periods of a subject in accordance with embodiments of the present disclosure.

FIGS. 15A, 15B, 15C, and 15D are marked-up heat maps of IMFs of EKG signals in accordance with some embodiments of the present disclosure.

### DETAILED DESCRIPTION OF THE PREFERRED EMBODIMENTS

It will be noted at the beginning that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

Several definitions that apply throughout this disclosure will now be presented.

The term “coupled” is defined as connected, whether directly or indirectly through intervening components, and is not necessarily limited to physical connections. The connection can be such that the objects are permanently connected or releasably connected. The term “comprising,” when utilized, means “including, but not necessarily limited to”; it specifically indicates open-ended inclusion or membership in the so-described combination, group, series and the like.

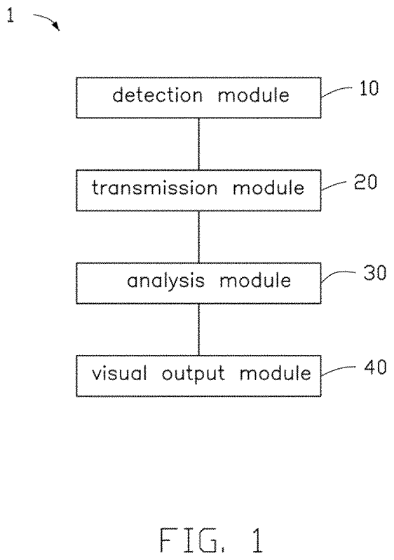

Referring to FIG. 1, a system for analyzing the physiological signals in accordance with an embodiment of the present disclosure is provided. The system 1 comprises a detection module 10, a transmission module 20, an analysis module 30 and a visual output module 40. The system 1 is configured to detect physiological signals, to analyze physiological signals and to display graphical information of the analyzed results. The physiological signal may include but not limited to: EKG signals, EMG signals, ERG signals, blood pressure, pulse oximetry signals, body temperature, and spirometry signals. It is contemplated that the system 1 may further comprise other electrical components or modules for better performance or user experience. For example, the system 1 may comprise an amplifier module or filter module to enhance signal to noise ratio by gaining signal strength within certain bandwidth and minimizing noise from environmental interference or baseline wandering. For example, the system 1 may comprise an analog-to-digital converter (ADC) for signal digitization. For example, the system 1 may further comprise a storage module for storing the digital signals or storing the analyzed data. In one example, the detection module 10 may further comprise a data acquisition module. The data acquisition module is capable of executing the functions of the amplifier module, ADC and the storage module. Furthermore, the system 1 may comprise a user input module for use to control the system 1, such as a keyboard, a mouse, a touch screen, or a voice control device.

The detection module 10 is configured to receive the physiological signals and to convert the physiological signals into electrical signal. The detection module 10 may convert cardiovascular activities, skeletal muscle activities, or blood pressure into electrical signals. The detection module 10 may comprise one or more sensing components, and the sensing component can be a transducer or a blood pressure meter. The transducer may be a biopotential electrode to detect the electrical potentials or a magnetoelectric transducer to detect the magnetic fields. The blood pressure meter may be an oscillometric monitoring equipment. It is contemplated that a ground electrode may be paired with the biopotential electrodes for measuring electrical potential differences and additionally a reference electrode may be presented for noise reduction. The detection module 10 may be applied on the surface of one or more specified regions of the living organism for the detection of specific physiological signals. The specified regions may include but not limited to: the chest for EKG, the skin above the skeletal muscle for EMG, or the skin above the vein for blood pressure. In one example, the detection module 10 comprises at least 10 biopotential electrodes being positioned on the limbs and the chest of the human body. In another example, the detection module 10 comprising an array of transducers may be arranged as a 10-20 system or other higher resolution systems. The biopotential electrodes could be wet (with saline water or conducting gels) or dry electrodes.

The transmission module 20 is configured to receive the electrical signals from the detection module 10 and deliver the signals to the analysis module 30. The transmission module 20 may be wired or wireless. The wired transmission module 20 may include an electrical conductive material delivering the detected signal directly to the analysis module 30 or to the storage module for processing by the analysis module 30 thereafter. The detected signal may be stored in a mobile device, a wearable device or transmitted wirelessly to a data processing station through RF transmitters, Bluetooth, Wi-Fi or the internet. The mobile device can be a smartphone, a tablet computer, or a laptop. The wearable device can be a processor-embedded wristband, a processor-embedded headband, a processor-embedded cloth, or a smartwatch. It is contemplated that the modules of the system 1 may be electrically coupled within a compact device or may be located discretely and coupled together by wired or wireless communication network.

The analysis module 30 is configured to process the signal by a series of steps. The analysis module 30 may be a single microprocessor, such as a general purpose central processing unit, an application specific instruction set processor, a graphic processing unit, a field-programmable gate array, a complex programmable logic device or a digital signal processor. The analysis module 30 comprises a non-transitory computer program product embodied in a computer-readable medium. The non-transitory computer program product can be a computer program, an algorithm, or codes that can be embodied in the computer-readable medium. The analysis module 30 may comprise multiple microprocessors or processing units to execute the non-transitory computer program product embodied in the computer-readable medium, in order to perform different functional blocks of the entire analysis process.

The visual output module 40 is configured to display the graphical results of the information generated by the analysis module 30. The visual output module 40 may be a projector, a monitor, or a printer for projecting the analysis results. In the examples, the analysis result is an visual output with graphic representations, and can be displayed by the visual output module 40 on a color monitor, be printed out on a paper or an electronic file, or be displayed on a grayscale monitor.

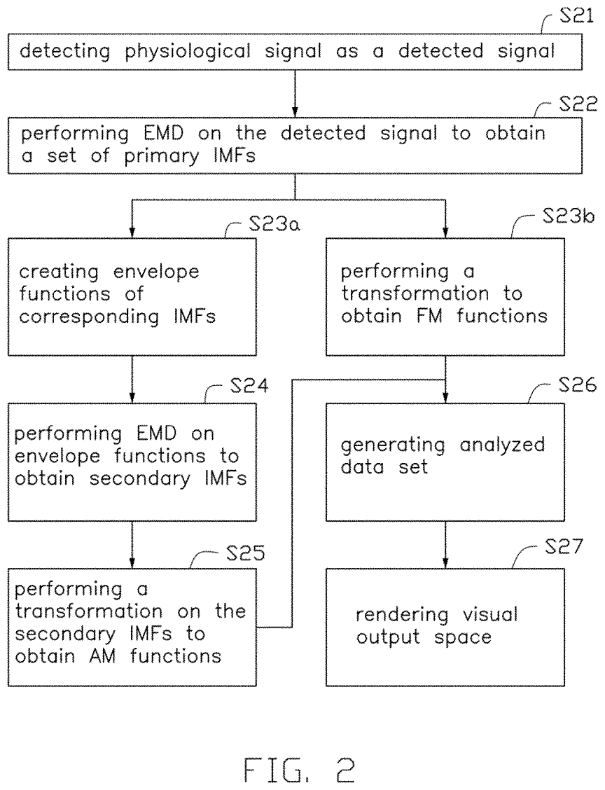

Referring to FIG. 2, a method for analyzing the physiological signals in accordance with an embodiment of the present disclosure is provided. The method for analyzing the physiological signals may include the steps as mentioned below. The method comprises: detecting the physiological signals as a detected signal S21, performing empirical mode decomposition (EMD) on the detected signal to obtain a set of primary intrinsic mode functions (IMFs) S22, creating envelope functions of the corresponding of IMF S23a, performing EMD on the envelope functions to obtain sets of secondary IMF S24, performing a transformation on the plurality of primary IMFs to obtain the frequency modulation (FM) functions S23b, performing a transformation on the plurality of secondary IMFs to generate the AM function S25, generating data set according to the FM function and the AM function S26, generating a visual output space S27. The EMD in S22 can be complete ensemble empirical mode decomposition (CEEMD), ensemble empirical mode decomposition (EEMD), masking EMD, enhanced EMD, multivariate empirical mode decomposition (MEMD), noise-assisted multivariate empirical mode decomposition (NA-MEMD). The transformation in S23b and S25 can be Hilbert transform, Direct quadrature, inverse trigonometric function, or generalized zero-crossing.

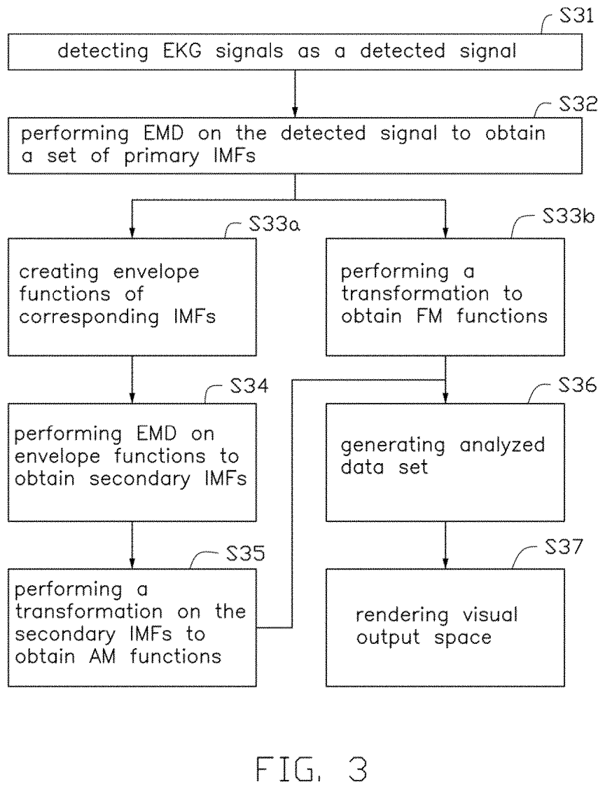

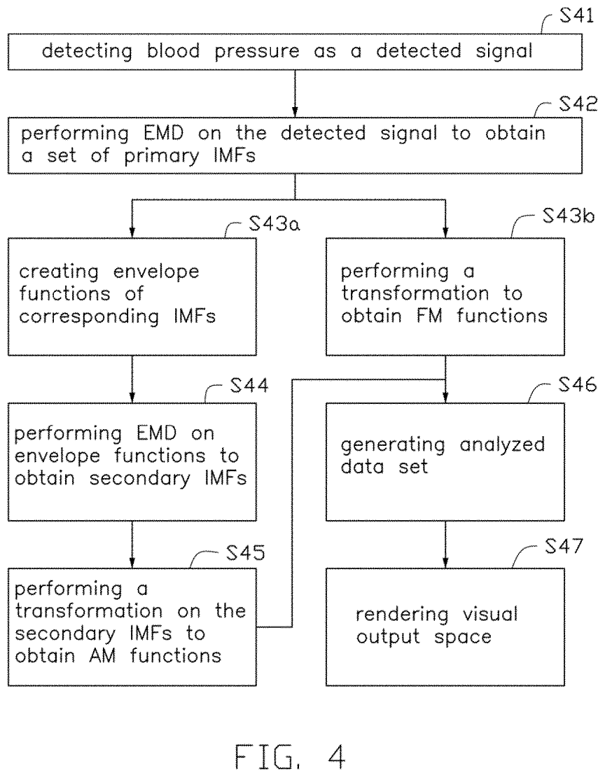

Detecting the physiological signals as one or more detected signals S21 is performed at the detection module. Referring to FIG. 3, the physiological signal may be EKG signals, in accordance with an embodiment of the present disclosure. Referring to FIG. 4, the physiological signal may be blood pressure, in accordance with an embodiment of the present disclosure. In one example, the detected signal may be acquired and stored by the data acquisition module in the form of electrical potential (preferably measured by voltage) with corresponding temporal sequences. The detected signal may be stored as a detected data set comprising a plurality of detected data units and each detected data unit comprises at least a signal strength and a time period. A sampling rate of the data acquisition module may determine a time interval of adjacent data. As illustrated in FIG. 1, the analysis module 30 generates the analyzed data set from the detected signal and the analyzed data set may be stored in the storage module for visual output module 40 thereafter. The analyzed data set comprises a plurality of analyzed data units.

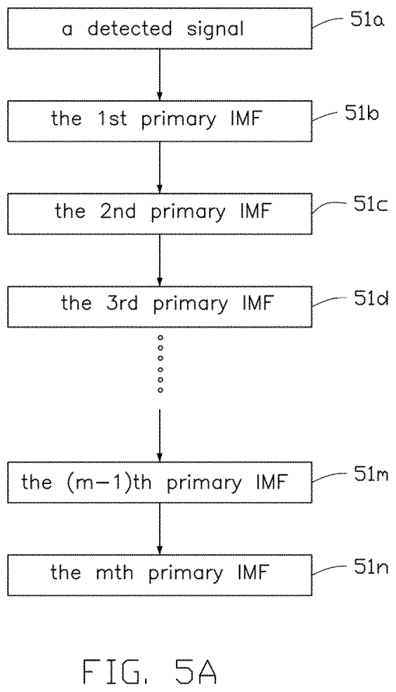

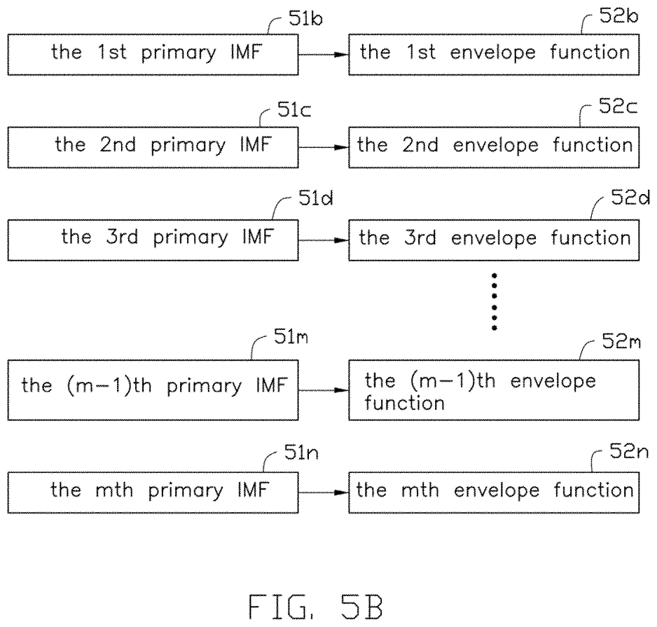

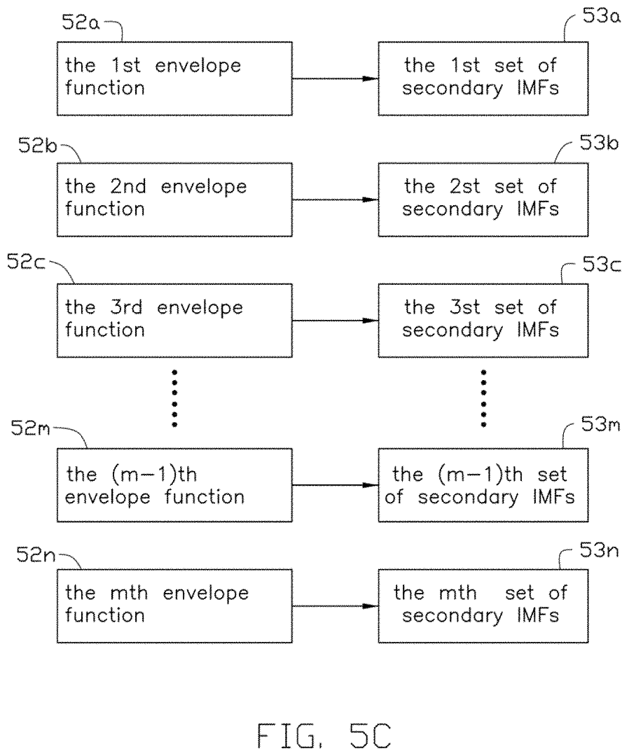

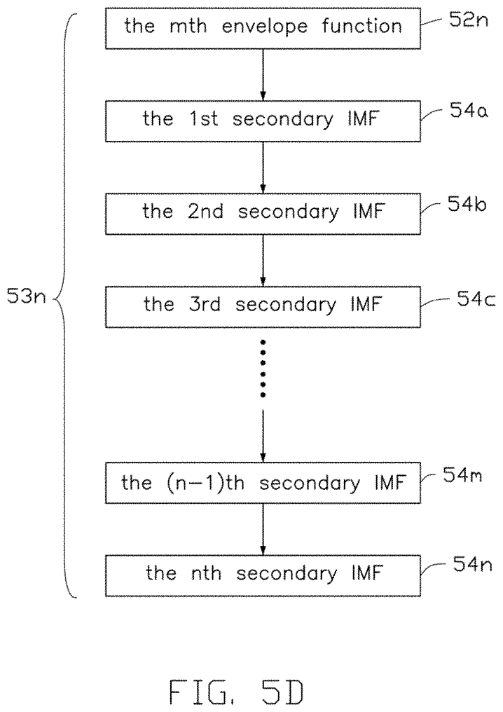

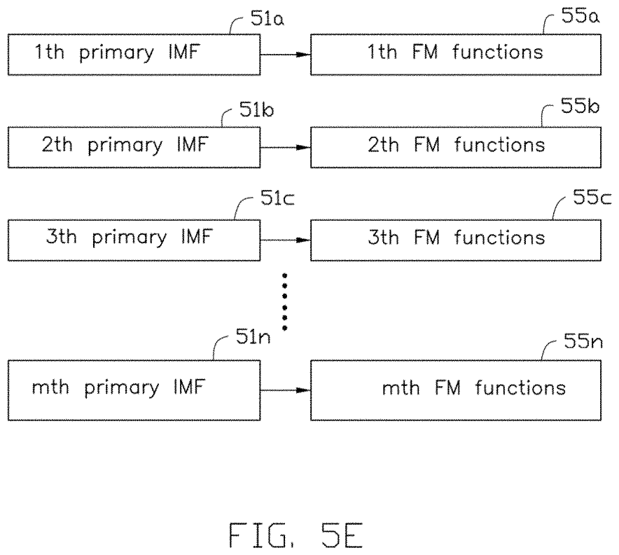

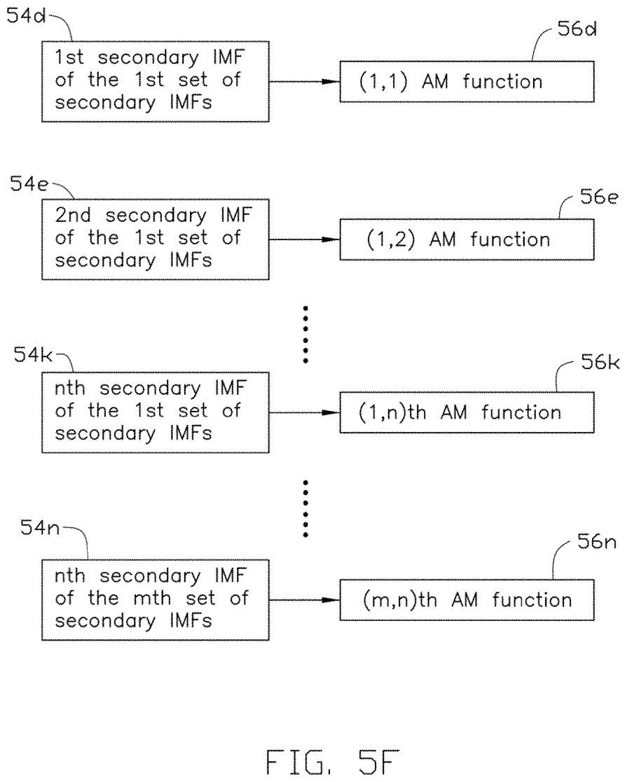

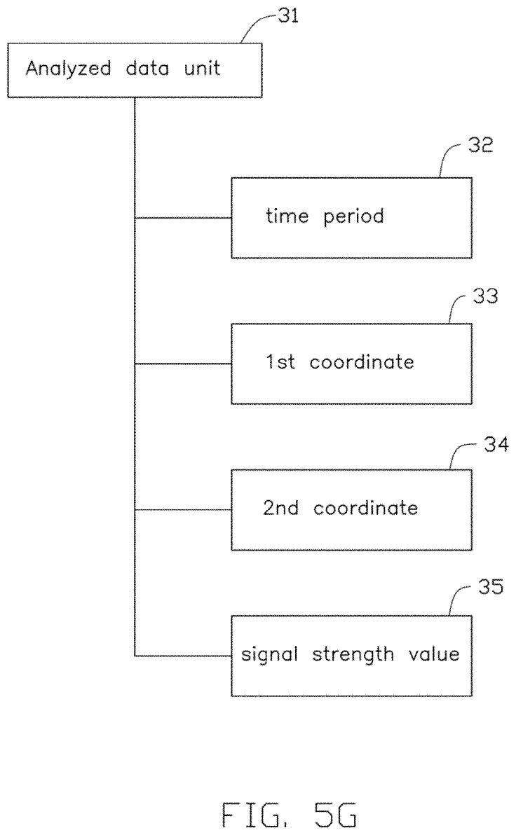

The processes S22, S23a, S23b, S25, S32, S33a, S33b, S35, S42, S43a, S43b, and S45 are further elaborated in FIG. 5A to FIG. 5F, in accordance with embodiments of the present disclosure. The detected signals are consequently transformed or decomposed into primary IMFs, secondary IMFs, envelope functions, AM functions, and FM functions.

Referring to FIG. 5A, a plurality of EMDs for detected signals are provided in accordance with an embodiment of the present disclosure. The detected signal is transformed into a set of primary IMFs by EMDs. The plurality of EMDs in FIG. 5A correspond to S22 of FIG. 2, S32 of FIG. 3, or S42 of FIG. 4. The EMD is a process comprising a series of sifting process to decompose a signal into a set of IMFs. For example, a plurality of primary intrinsic functions is generated from the detected signal by EMD. A sifting process generates an intrinsic function from the detected signals. For example, a first sifting process generates a first primary IMF 51b from the detected signal 51a; a second sifting process generates a second primary IMF 51c from the first primary IMF 51b; a third sifting process generates a third primary IMF 51d from the second primary IMF 51c; a mth sifting process generates a mth primary IMF 51n from the (m−1)th primary IMF 51m. The number of sifting processes is determined by stopping criteria. The stopping criteria may depend on the signal attenuation or the variation of the mth primary IMF 51n.

Furthermore, EMD may comprise masking procedure or noise (even pairs of positive and negative values of the same noise) addition procedure with variable magnitude adapted for each sifting step to solve mode mixing problems. It is contemplated that EMD may be achieved by ensemble techniques.

Referring to FIG. 5B, a plurality of interpolation processes is provided in accordance with an embodiment of the present disclosure. The interpolation processes in FIG. 5B correspond to S23a in FIG. 2, S33a in FIG. 3, or S43a in FIG. 4. An envelope function is the interpolation function generated by an interpolation process from detected signals. The envelope function connects local extrema of the detected signals. Preferably, the envelope connects the local maxima of the absolute-valued function of the detected signals. The interpolation process may be achieved via linear interpolation, polynomial interpolation, trigonometric interpolation or spline interpolation, preferably cubic spline interpolation. The envelope functions in FIG. 5B are generated from IMFs in FIG. 5A by the interpolation processes. A first envelope function 52a may be generated from the first primary IMF 51a; a second envelope function 52b may be generated from the second primary IMF 51b; a third enveloped function 53b may be generated from the third primary IMF 53a; a (m−1)th envelope function 52m may be generated from the (m−1)th primary IMF 51m; a mth envelope function 52n may be generated from the nth primary IMF 51n.

Referring to FIG. 5C, a plurality of EMDs is provided in accordance with an embodiment of the present disclosure. The plurality of sets of secondary intrinsic functions are generated from the envelope functions by EMD. The EMDs in FIG. 5C correspond to S24 in FIG. 2, S34 in FIG. 3, or S44 in FIG. 4. The first set of secondary IMFs 53a is generated from the first envelope function 52a; the second set of secondary IMFs 53b is generated from the second envelope function 52b; the (m−1)th set of the plurality of secondary IMFs 53m is generated from the (m−1)th envelope function 52m; the mth set of the plurality of secondary IMFs 53n is generated from the mth envelope function 52n.

Referring to FIG. 5D, a plurality of sets of secondary IMFs are provided in accordance with an embodiment of the present disclosure. The mth envelope function 52n, the mth set of secondary IMFs 53n, and the secondary IMFs included in the mth set of secondary IMFs 53n are illustrated in FIG. 5D. The mth envelope function 52n in FIG. 5B comprises a first secondary IMF 54a of the mth set of secondary IMFs 53n, a second secondary IMF 54b of the mth set of secondary IMFs 53n, a third secondary IMF 54c of the mth set of secondary IMFs 53n, a (n−1)th secondary IMF 54m of the mth set of secondary IMFs 53n, and a nth secondary IMF 54n of the mth set of secondary IMFs 53n. Therefore, there are IMFs in a number of m (number of the plurality of sets of secondary IMF) multiplying n (number of individual secondary IMFs in a set of secondary IMF in FIG. 5D.

Referring to FIG. 5E and FIG. 5F, a series of transformation processes is provided in accordance with an embodiment of the present disclosure. The transformation process is to convert a function from real domain to complex domain. The transformation process comprises at least a transformation and a complex pair function formation. The transformation process may be a Hilbert transform, a direct-quadrature-zero transform, an inverse trigonometric function transform, or a generalized zero-crossing transform. The complex pair function formation is to combine the function as the real part of the complex pair function and the transformed function as the imaginary part of the complex pair function.

In FIG. 5E, the FM functions are the complex pair functions generated from the plurality of primary IMFs by a proper transformation process. The transformation processes in FIG. 5E correspond to S23b in FIG. 2, S33b in FIG. 3, or S43b in FIG. 4. The first primary IMF 51a is transformed into a first FM function 55a by the transformation process; the second primary IMF 51b is transformed into a second FM function 55b by the transformation process; the third primary IMF 51c is transformed into a third FM function 55c by the transformation process; and the mth primary IMF 51n is transformed into a mth FM function 55n by the transformation process.

In FIG. 5F, the AM functions are the complex pair functions generated from the secondary IMFs by a series of transformation processes. The transformation processes in FIG. 5F correspond to S25 in FIG. 2, S35 in FIG. 3, or S45 in FIG. 4. The first secondary IMF 54d of the first set of secondary IMFs may be transformed into a (1,1) AM function 56d by the transformation process; the second secondary IMF 54e of the first set of secondary IMFs is transformed into a (1,2) AM function 56e by the transformation process . . . and the nth secondary IMF 54k of the first set of the secondary IMFs is transformed into a (1, n) AM function 56k by the transformation process. Furthermore, the nth secondary IMF 54n of the mth set of secondary IMFs may be transformed into a (m, n)th AM function 56n by the transformation process.

Referring to FIG. 5G, components of an analyzed data unit is provided in accordance with an embodiment of the present disclosure. In FIG. 5G, the analyzed data unit 31 comprises a time period 32, a first coordinate 33, a second coordinate 34 and a signal strength value 35. In one embodiment, the time period 32 is a period of time when the detection module detects the physiological signals, the first coordinate 33 indicates instantaneous frequency of FM measured by frequency (Hertz), and the second coordinate 34 indicates instantaneous frequency of AM measured by frequency (Hertz). The signal strength value 35 may indicate signal amplitude measured by electrical potential (voltage) or electrical current (ampere) or may indicate signal energy measured by energy strength per unit time interval (watt). For each analyzed data unit within the time period, the first coordinate 33 can be the argument of the mth FM functions 55n in FIG. 5E at corresponding time period; the second coordinate 34 can be the argument of the (m, n)th AM function 56n in FIG. 5F at corresponding time period; the signal strength value 35 is the value of the envelope function at corresponding time period. Preferably, the second coordinate 34 is larger than the first coordinate 33.

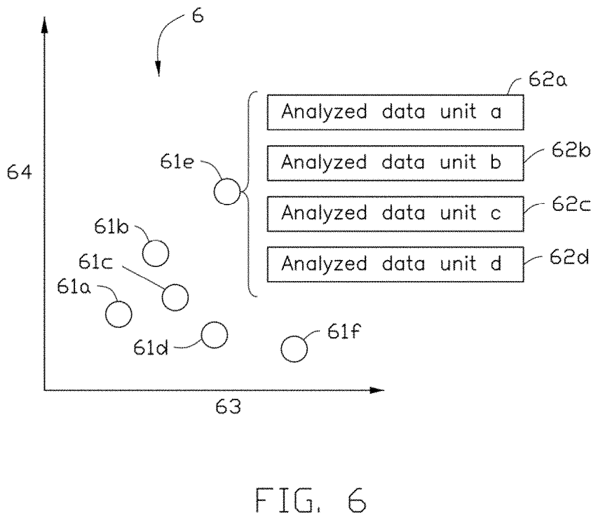

Referring to FIG. 6, a schematic visual output from a plurality of analyzed data sets is provided in accordance with an embodiment of the present disclosure. In FIG. 6, the visual output 6 comprises a first axis 63, a second axis 64 and a plurality of other visual elements 61a-61f. The first axis 63 can be a frequency scale of FM, or a logarithmic scale of FM. The second axis 64 can be a frequency scale of AM, or a logarithmic scale of AM. Each of the visual elements 61a-61f comprises an analyzed data set and an accumulated signal strength. Each of the analyzed data set is an integral of a plurality of analyzed data units, therefore each of the visual element 61a-61f comprises multiple analyzed data units within a certain range of the FM frequency and the AM frequency in a time period. The accumulated signal strength of each of the visual elements 61a-61f is an integral of the signal strengths of each of the analyzed data units. For instance, the accumulated signal strength of the visual element 61e is an integral of the signal strengths of the analyzed data unit a 62a, the analyzed data unit b 62b, the analyzed data unit c 62c, and the analyzed data unit d 62d. The accumulated signal strength can be presented by color scale, dot density, grayscale, or screetones, wherein different colors, dot densities, grayscales, or screetones indicate different values of the accumulated signal strength (not shown). The visual output module 40 in FIG. 1 renders a visual output space according to the analyzed data sets, and displays the visual output 6.

It is contemplated that a smoothing process may be applied to the visual output space for the visual elements with sparse data units. For example, the smoothing process may be Butterworth filter, exponential smoothing, Kalman filter, Kernal smoother, Laplacian smoothing, moving average, or other image smoothing techniques.

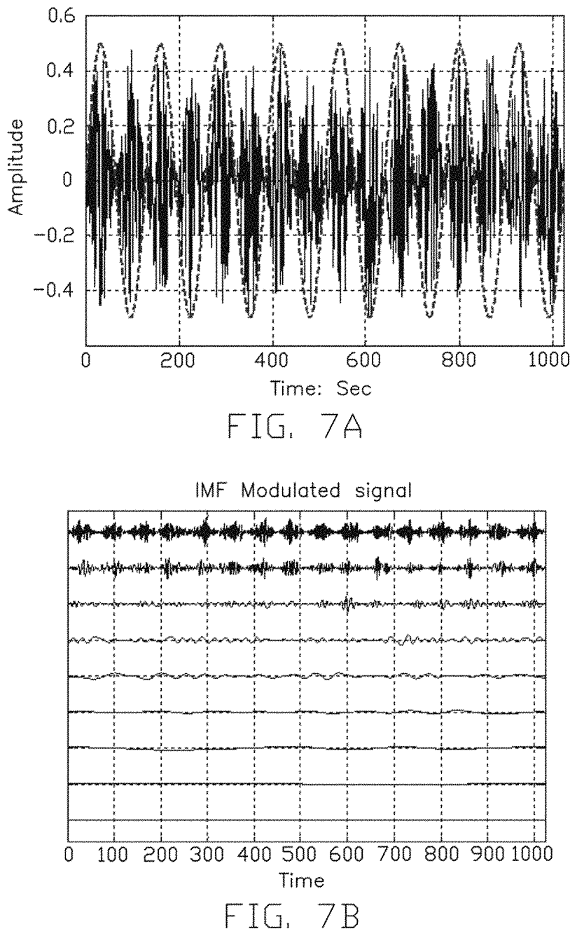

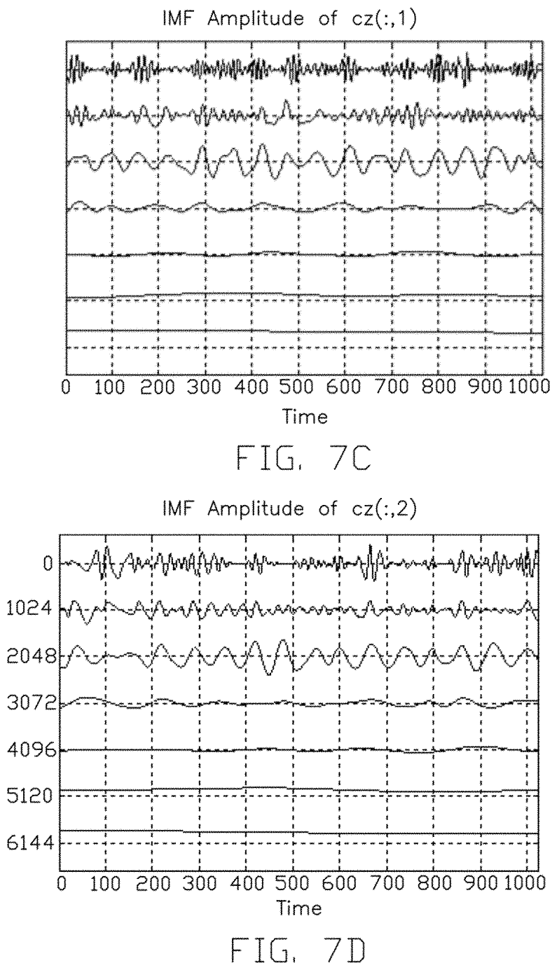

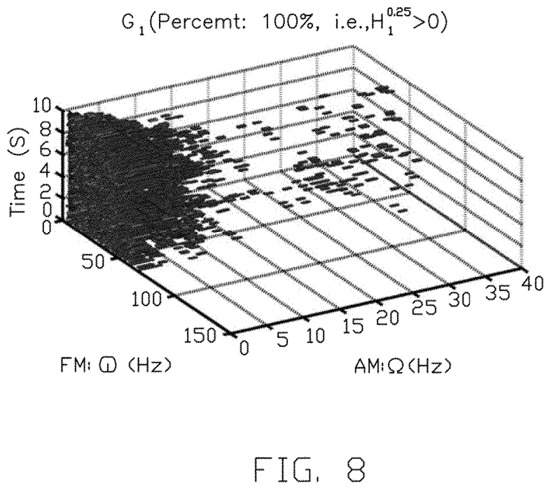

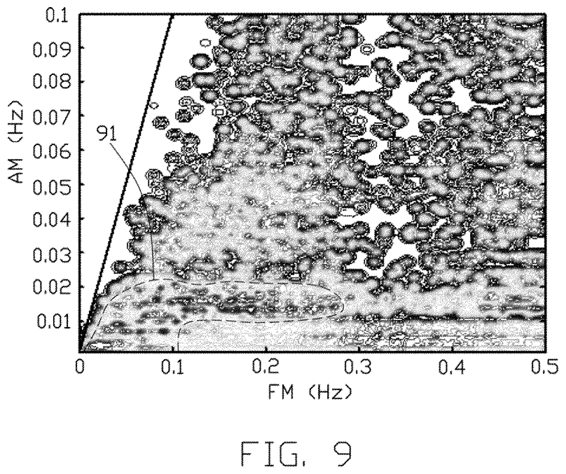

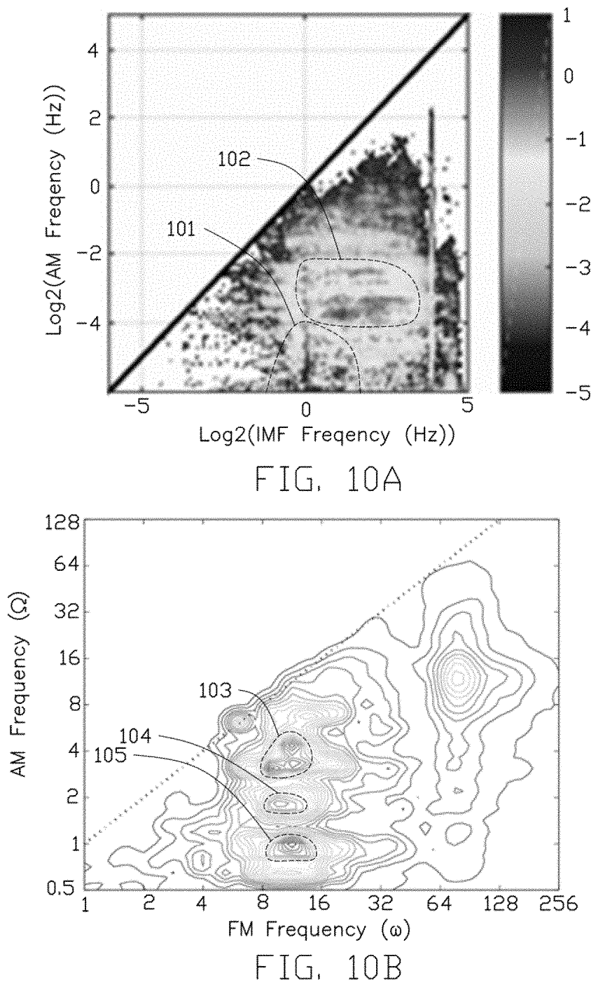

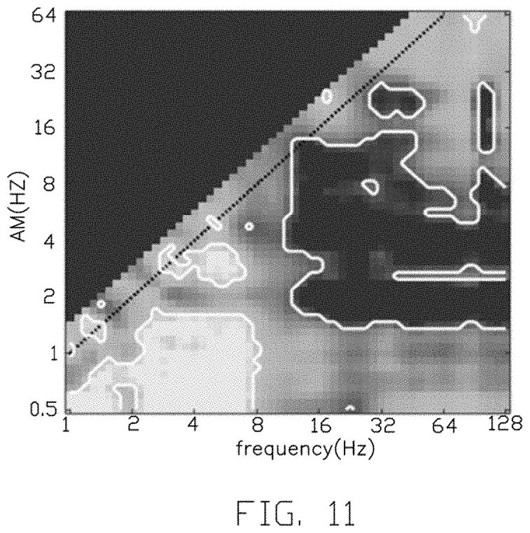

Following the methods, principles and transformation processes illustrated in FIG. 2, FIG. 5A-5G, and FIG. 6, a plurality of embodiments from detected physiological signals are demonstrated in FIG. 7A-7D, FIG. 8, FIG. 9 FIG. 10A-10B, and FIG. 11.

The detected signal and the IMFs generated via EMD process are shown in FIG. 7A-7D in accordance with an embodiment of the present disclosure. In some embodiments, FIG. 7A-7D are intermediate outputs from the detected physiological signals. In FIG. 7A, the detected signal stored as the detected data set is plotted along time. FIG. 7B shows a plurality of primary IMFs generated from the detected signal by EMD. FIG. 7C shows the first set of secondary IMFs generated from the first envelope function. FIG. 7D shows the second set of secondary IMFs generated from the second envelope function.

Referring to FIG. 8, a visual output for the analyzed data units are provided in accordance with an embodiment of the present disclosure. The analyzed data units are plotted in a three-dimensional space comprising AM axis, FM axis, and time axis. The signal strength of each of the analyzed data units is also given but not shown in the visual output. Each plotted dot is an analyzed data unit. An integral of analyzed data units within a time period is an analyzed data set.

Referring to FIG. 9, a heat map transformed from the plot graph of FIG. 8 is provided in accordance with an embodiment of the present disclosure. The heat map is a form of the visual output. The heat map comprises an axis of FM and an axis of AM. In the embodiment, each visual element comprises an analyzed data sets and an accumulated signal strength, which is an integral of all the signal strength of the analyzed data units within a time period. In other words, the time axis of FIG. 8 is deducted in FIG. 9. As illustrated in FIG. 9, the grayscale represents the accumulated signal strength, and may have different shades of gray proportional to the accumulated signal strength: a dark gray or black to represent the smallest accumulated signal strength, a lighter gray surrounded by a darker gray to represent an intermediate accumulated signal strength, and a dark gray surrounded by a lighter gray to represent the largest accumulated signal strength. For instance, an area 91 is a combination of an area with 0-0.3 Hz of FM and 0.01-0.02 Hz of AM and another area with 0-0.1 Hz of FM and 0-0.01 Hz of AM. The area 91 is an area of dark gray surrounded by lighter gray, so the area 91 has the largest accumulated signal strength in FIG. 9. Conversely, the grayscale may also use a dark gray surrounded by a lighter gray to represent the smallest accumulated signal strength, and a dark gray or black to represent the largest accumulated signal strength in some embodiments.

Additionally, the accumulated signal strength in the heat map may be represented by a color scale, dot density, or screentone. In one embodiment, the dot density may be higher for a larger accumulated signal strength, and lower density for a smaller accumulated signal strength. In another embodiment, the color scale may use blue to indicate the smallest accumulated signal strength, green to indicate an intermediate accumulated signal strength, and yellow, orange, or red to indicate the largest accumulated signal strength. The color scale may also include a color transition from one color to another color, such as the color transition from blue to green or from orange to red. In still another embodiment, the screentone with more grids may represent larger accumulated signal strength, and the screentone with more dots may represent lower accumulated signal strength. Conversely, the color scale, dot density, or screentone can have different meanings for different colors, dot densities, contour lines, or screentones for various levels of the accumulated signal strength.

The dot densities in dot density graph, different shades of gray in grayscale, various colors in the color scale, the densities of contour lines, and different screentones in the visual output indicate the accumulated signal strength by the analyzed data unit, and they may represent a relative or an absolute scale of the accumulated signal strength. It is contemplated that the visual output space may be rendered dynamically along with sliding time periods so that the visual output module is capable of displaying the HOSA spectrum not only as a graph, but as a video.

Referring to FIG. 10A and FIG. 10B, visual outputs of a logarithmic scale of AM axis and FM axis of the analyzed data unit are provided in accordance with embodiments of the present disclosure. In FIG. 10A, the X-axis is a logarithmic scale of FM, and the Y-axis is a logarithmic scale of AM. The accumulated signal strengths in FIG. 10A is indicated by a grayscale, which has similar meanings as the grayscale in FIG. 9. An area 101 and another area 102 have the largest accumulated signal strengths in FIG. 10A. The area 101 is generally located in Log 2 FM=0 and Log 2 AM=−6 to −4, and the area 102 is generally located in Log 2 FM=0-5 and Log 2 AM=−4 to −2. Preferably, the base of the logarithmic scale is assigned as 2 because of the dyadic property of EMD process. In FIG. 10B, the HOSA spectrum may be plotted with contour lines, with higher density of the contour lines representing larger accumulated signal strength. For instance, an area 103, an area 104, and an area 105 have the largest accumulated signal strengths in FIG. 10B. The area 103 is generally located in 4 Hz of AM and 8-16 Hz of FM. The area 104 is generally located in 2 Hz of AM and 8-16 Hz of FM. The area 105 is generally located in 1 Hz of AM and 8-16 Hz of FM. The contour lines may also be combined with dot density, color scale, or screentone to indicate various levels of the accumulated signal strength.

Referring to FIG. 11, another visual output of an AM axis and a FM axis with enhanced contrast is provided in accordance with an embodiment of the present disclosure. FIG. 11, the visual elements, or the blocks, may represent the difference between an analyzed data unit and a reference data unit. The contrast may be processed by a normalization process to align with a linear scale or a distribution model, such as normal distribution. The reference data units are used as control group data, and may be generated from a standard data unit or a longitudinal data unit. The standard data unit is generated from an average of the analyzed data units from a specific group of subjects. For example, the specific group of subjects may be healthy subjects or subjects diagnosed without a particular disease state. To eliminate the individual variations, normalization of the individual data can be used. The longitudinal data unit is an experiment group, and may be generated from the previous analyzed data units of the same subject. In some embodiments, z-score can be calculated according to the reference dataset. The device may further generate a graph to demonstrate that the location of the analyzed data unit in a distribution model.

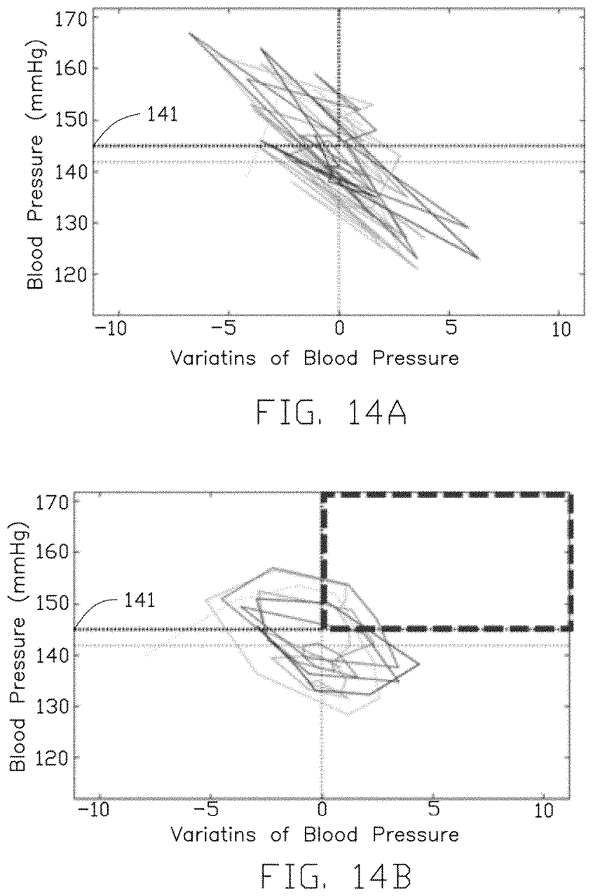

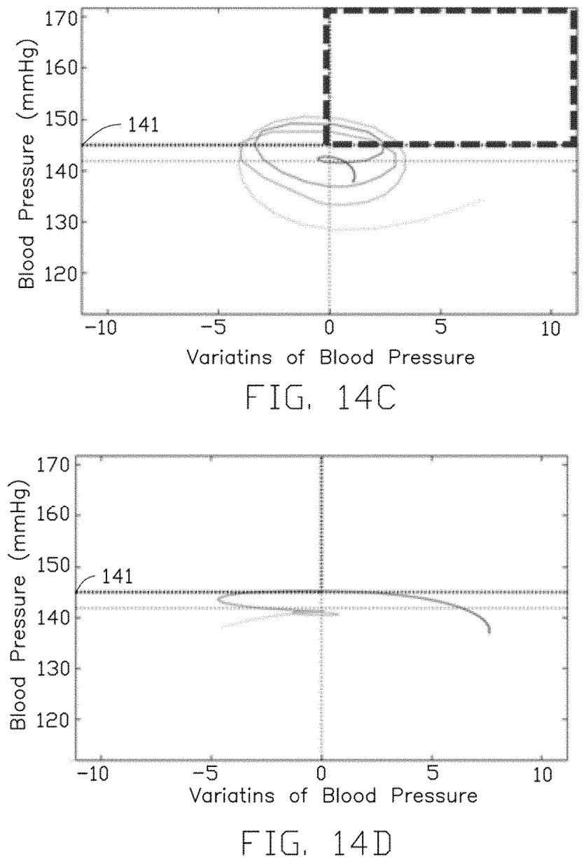

The visual output of the analyzed data set of the physiological signals can be used to compare two or more states of different groups of people, different individuals, or the same individual. The visual output can be the heat map as in FIG. 9, the logarithmic graph of AM and FM as in FIG. 10A-10B, the non-logarithmized AM-FM graph of FIG. 11, or a trajectory graph of the signal strength value and the variations of the signal strength as in FIG. 14A-14B. Specific patterns of one or more particular diseases can be identified. The specific patterns may comprise a disease state, a healthy state, a good prognosis state, a poor prognosis state, or other patterns relevant to diagnosis, prognosis, clinical evaluation, or staging of the disease. The comparison between the specific patterns may be used to identify the difference between two groups of people with different health condition, two groups of people with different disease stage, two groups of people with different prognosis of the disease, two individuals with different health condition, two individuals with different disease stage, two individual with different prognosis of the disease, or two different time periods of the same individual.

The comparison on specific patterns may lead to establish a model for the clinical evaluation, diagnosis, staging or prognosis of the disease. The differences identified by the comparison can be quantitative. A set of probabilities for quantifying statistical significance can be generated from two visual outputs, with each visual output representing a group of peoples, a disease stage, a prognosis of the diseases, an individual, or a health condition. The probability can be a P-value, or other statistical analyses. The probability distribution can be presented by a probability visual output. The visual output comprises a first axis of a logarithmic scale of FM, a second axis of a logarithmic scale of AM, and a plurality of visual elements defined by the first axis and the second axis. Each of the visual elements comprises a probability for quantifying statistical significance. The visual output is generated by a visual output module. The visual output module renders a visual outputs space according to the set of probabilities, and the set of probability is generated by an analysis module according to two different visual outputs. The visual output can be an intuitive visualization for the comparison between two other visual outputs.

Another tool for comparing two visual outputs can be area-under-curves (AUCs). A set of AUCs are generated from comparisons of the logarithmic IMFs between two or more states of different groups of people, different individuals, or the same individual. AUCs can be presented by an AUC visual output. The AUC visual output comprises a first axis of a logarithmic scale of FM, a second axis of a logarithmic scale of AM, and a plurality of AUC visual elements defined by the first axis and the second axis. Each of the AUC visual elements comprises an AUC. The AUC visual output is generated by a visual output module. The visual output module renders a visual output space according to the set of AUCs, and the set of AUC is generated by an analysis module according to two different visual outputs. The AUC visual output can also be an intuitive visualization for the comparison between two other visual outputs.

A healthy state could be defined as a subject or a group of subjects without being diagnosed with particular disease(s) of interest. A disease state could be defined as a subject or a group of subject being diagnosed with particular disease(s) of interest. The healthy state and the disease state may be presented on the same subject on different time periods or be presented on different subjects. A relevancy of the particular disease(s) of interest and the physiological signals detected can be well-known: the relevancy may be that the specific physiological signals are needed for the diagnosis of the particular disease of interest, or the physiological signals are specifically identified in the patients of the particular disease of interest. The relevancy may include but not limited to: EKG signals for heart diseases (e.g. ischemic heart disease, hypertensive heart disease, rheumatic heart disease, inflammatory heart disease), blood pressure for vascular diseases, pulse oximetry signals for vascular diseases or anemia, body temperature for inflammatory diseases, or spirometry signals for respiratory diseases.

The present disclosure will now be described more specifically with reference to the following exemplary embodiments, which are provided for the purpose of demonstration rather than limitation.

### Example 1: Visualization and Assessment of Blood Pressure

Referring to FIG. 12, a graph of blood pressure of a subject is provided in accordance with an embodiment of the present disclosure. The blood pressure of a subject is measured twice a day by a system of analyzing blood pressure. In FIG. 12, the X-axis represents time and the Y-axis represents pressure units of mmHg, with diastolic pressure being the lower curve and systolic pressure being the upper curve.

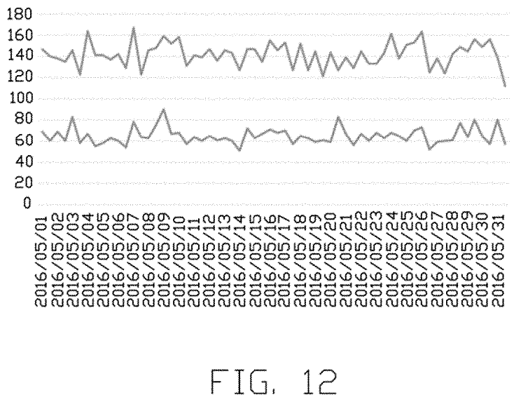

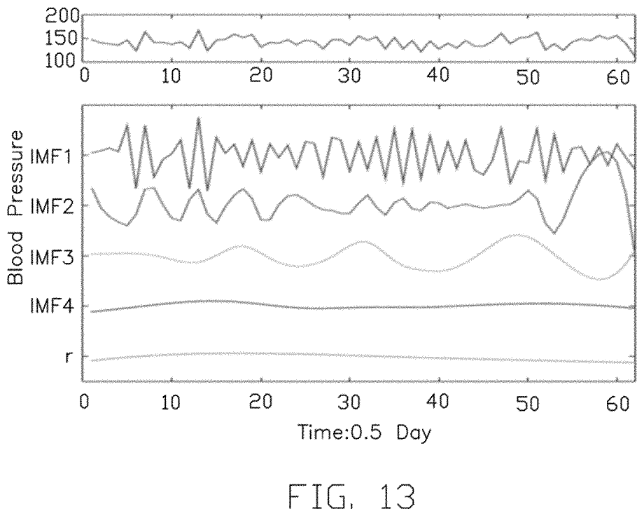

Referring to FIG. 13, an IMF modulated signal graph of blood pressure is provided in accordance with an embodiment of the present disclosure. The detected signals from blood pressure are in the upper block. The detected signals from blood pressure are then transformed into intrinsic mode functions (IMFs) via the empirical mode decomposition (EMD) processes, as shown in FIG. 5A-5D. The IMF1 in the lower block is generated from the detected data of the blood pressure via EMD process as shown in FIG. 5A. The IMF2 are generated via EMD from the IMF1. The IMF3 and IMF4 in the lower block are generated consecutively by EMD process as in FIG. 5B-5D, and are similar to the sets of the IMFs in the intermediate outputs in FIG. 7B-7D.

A system can be used for generating the IMF1-IMF4. The system comprises a detection module for detecting blood pressure, a transmission module for receiving the detected signals of the blood pressure from the detection module and delivering the detected signals to an analysis module, and a non-transitory computer program product. When executing the non-transitory computer program product, the analysis module performs the following actions: 1) calculating the variation of blood pressure, which is the variation of detected signals within a time period; 2) combining the variations of the blood pressure and the blood pressure to generate a primary analyzed data set. The actions performed by the analysis module further comprises: 3) performing EMD on the detected signals of the blood pressure to generate the IMF1, the IMF2, the IMF3, and the IMF4; 4) calculating variations of IMF, and the variation of the IMFs is the variation of an IMF within a time period; 5) combining the variations of the IMF1-IMF4 and the IMF 1-IMF4 to generate a plurality of secondary analyzed data sets. The system further comprises a visual output module for rendering a visual output space according to the primary analyzed data set and the secondary analyzed data sets, and displaying a visual output. A threshold value can be input to the visual output module or the analysis module to indicate clinical importance in a cardiovascular disorder.

Referring to FIG. 14A-14D, variations of blood pressure are provided in accordance with an embodiment of the present disclosure. The detected signals are combined with the variation of the blood pressure to form a primary analyzed data set, and being presented as a trajectory in the visual output space. The IMF1 are combined with the variation of the IMF1, the IMF2 are combined with the variation of the IMF2, the IMF3 are combined with the variation of the IMF3, and the IMF4 are combined with the variation of the IMF4, and the above four combinations are secondary analyzed data sets being presented by four trajectories in the visual output spaces. In FIG. 14A-14D, the Y-axis represents detected data of the blood pressure (mmHg) and the X-axis represents variations of blood pressure and the IMFs. FIG. 14A is the variation of blood pressure of the subject within a 1.2-day-period. FIG. 14B is the variation of blood pressure of the subject within a 2.9-day-period. FIG. 14C is the variation of blood pressure of the subject within a 7.4-day-period. FIG. 14D is the variation of blood pressure of the subject within a 13.2-day-period. A threshold value 141 for blood pressure can be indicated and located on a midpoint of the Y-axis. The threshold value 141 can be a value of significant clinical importance in the cardiovascular disorder, and can be adjusted to reflect one or more criteria on the clinical evaluation, diagnosis, prognosis, or staging of other diseases.

A combination of detected data of the blood pressure and the variation of the blood pressure can be a model for clinical evaluation, a reference, an assessment, or an alert signal used for the diagnosis, prognosis, or staging for a cardiovascular disease. In FIG. 14A-14D, the threshold values 141 are set to 145 mm Hg. Zero variation can be indicated and located on a midpoint on the X-axis. Four quadrants can be divided by the threshold value on the Y-axis and zero on the X-axis, whereby the upper-right portion indicates when the blood pressure is over than 145 mm Hg and the variation of the blood pressure is larger than 0, and the lower-left portion indicates when the blood pressure is lower than 145 mm Hg and the variation of the blood pressure is smaller than 0. There is a higher possibility of sudden elevation of blood pressure if trajectories are presented in the upper-right portion of FIG. 14A-14D, and the subject may have a higher risk of developing a sudden elevation of blood pressure. The subject may receive hypertension alert signals after such trajectory is presented. On the other hand, there is a higher possibility of sudden decrease of blood pressure if trajectories are presented in the lower-left portion of FIG. 14A-14D, and the subject may have a higher risk of developing a sudden decrease of blood pressure. The subject may receive hypotension alert signals after such trajectory is presented.

In some embodiments, the X-axis in FIG. 14A-14D can be a scale of arguments of the variations of the IMFs. In some other embodiments, the X-axis can be a logarithmic scale of arguments of the variations of the IMFs, and the Y-axis can be a logarithmic scale of the signal strength of the IMFs.

### Example 2: Visualization and Assessment of EKG Signals

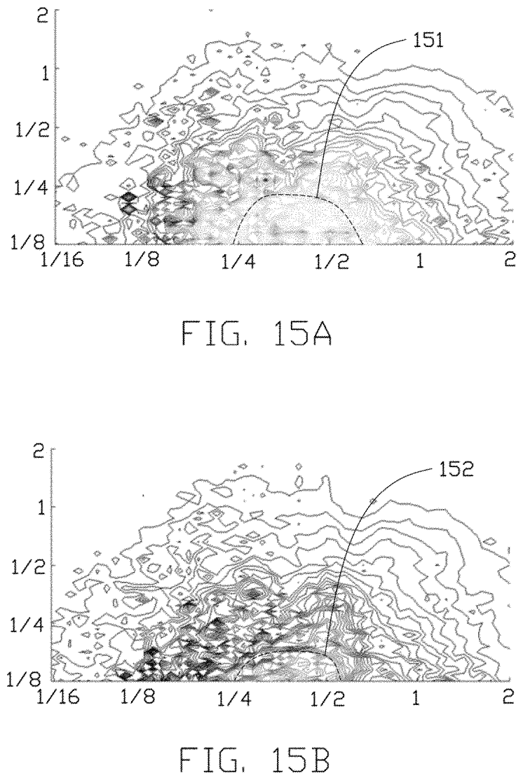

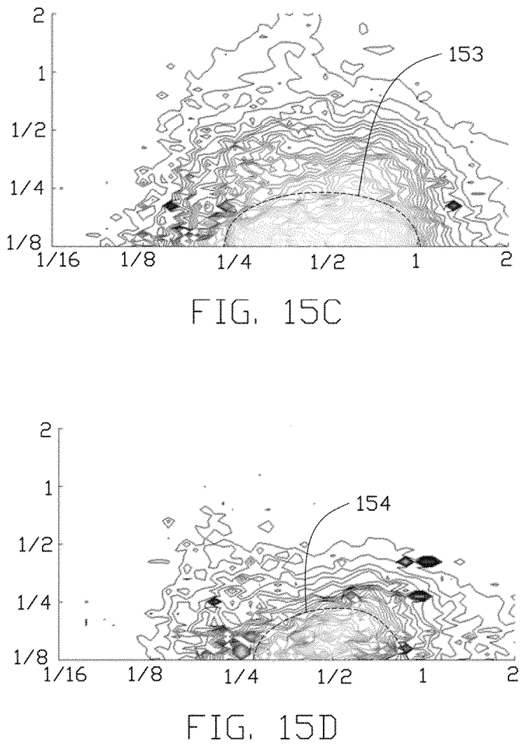

Referring to FIG. 15A-15D, heat maps transformed from plot graph of intrinsic mode functions (IMFs) are provided in accordance with an embodiment of the present disclosure. The heat maps of FIG. 15A-15D are presented in contour lines whereby higher density of contour lines represents larger accumulated signal strengths. The heat maps of FIG. 15A-15D are generated from data sets of IMFs. The IMFs are modulated from EKG signals detected in young or elder healthy subjects, subjects diagnosed with congestive heart failure (CHF), and subjects with liver transplantation, in a time period. The IMFs are generated via the empirical mode decomposition (EMD) process, as shown in FIG. 5A-5D. The heat maps in FIG. 15A-15D comprise a Y-axis as amplitude modulation (AM) and an X-axis as frequency modulation (FM), and are similar to visual outputs in FIG. 9. Each visual element in the heat maps in FIG. 15A-15D comprises an analyzed data set, which is an integral of all the analyzed data units within a time period. The visual elements in the heat maps in FIG. 15A-15D further comprise accumulated signal strengths. The accumulated signal strength is indicated by a grayscale, with lighter gray with contrast lines represents the largest accumulated signal strength and dark gray represents the smallest accumulated signal strength.

The heat map of FIG. 15A is the AM-FM distribution of the IMFs from the group of healthy young subjects. The heat map of FIG. 15B is the AM-FM distribution of the IMFs from the group of healthy elder subjects. The heat map of FIG. 15C is the AM-FM distribution of the IMFs from the group of subjects with CHF. The heat map of FIG. 15D is the AM-FM distribution of the IMFs from the group of subjects with liver transplantation. When comparing the distribution of accumulated signal strength in FIG. 15A-15D, the heat map of FIGS. 15A and 15B have the largest accumulated signal strength in an area 151 and an area 152, which are generally located in ¼-½ Hz of FM and ¼-⅛ Hz of AM, but the largest accumulated signal strength in FIGS. 15C and 15D are in an area 153 and an area 154, which are generally located in ¼-1 Hz of FM and ¼-⅛ Hz of AM. Moreover, the heat map of FIG. 15A and FIG. 15B are of higher complexity when comparing with the heat map of FIG. 15C and FIG. 15D.

Specific patterns of the AM-FM distributions in FIG. 15A-15D can be linked to cardiovascular diseases. Additional comparison between each of FIG. 15A-15D may be required to evaluate their differences. For instance, FIG. 15A-15D may be logarithmized, and the statistical significance between at any two of the above figures can be quantified. The P-value or other statistical analysis are applicable for the quantification The comparison on specific patterns may lead to establish a model for the clinical evaluation, diagnosis, staging or prognosis of cardiovascular diseases.

The heat maps of FIG. 15A-15D present different perspectives of EKG information among different groups of subjects, and are more concise and useful than raw EKG data. Based on the same amount of detected EKG data utilized in FIG. 15A-15D, conventional EKG graph would be overwhelming for professionals to analyze and diagnose.

Previous descriptions are only embodiments of the present disclosure and are not intended to limit the scope of the present disclosure. Many variations and modifications according to the claims and specification of the disclosure are still within the scope of the claimed disclosure. In addition, each of the embodiments and claims does not have to achieve all the advantages or characteristics disclosed. Moreover, the abstract and the title only serve to facilitate searching patent documents and are not intended in any way to limit the scope of the claimed disclosure.

What is claimed is:

1. A non-transitory computer program embodied in a computer-readable medium which, when executed by one or more analysis modules, provides a visual output for presenting physiological signals, comprising:
a first axis representing frequency modulation (FM);
a second axis representing amplitude modulation (AM); and
a plurality of visual elements, each of the visual elements being defined by the first axis and the second axis, and each of the visual elements comprising an accumulated signal strength and a plurality of analyzed data units from a time period,
wherein each of the analyzed data units comprises a first coordinate, a second coordinate, and a signal strength value, the first coordinate is an argument of a FM function from a transformation on a primary intrinsic mode function (IMF), the second coordinate is an argument of an AM function from a transformation on a secondary IMF, each of the primary IMF is generated from an empirical mode decomposition (EMD) of a plurality of the physiological signals, and each of the secondary IMF is generated from an EMD of the primary IMF, and the accumulated signal strength is an integral of the signal strength value of each of the analyzed data units.

2. The non-transitory computer program product of claim 1, wherein the first axis is a logarithmic scale of frequency modulation (FM), the second axis is a logarithmic scale of amplitude modulation (AM), the first coordinate is a logarithmic value of the argument of the FM function, and the second coordinate is a logarithmic value of the argument of the AM function.

3. The non-transitory computer program product of claim 1, wherein the physiological signals are electrocardiogram (EKG) signals, electromyogram (EMG) signals, electroretinography (ERG) signals, blood pressure, pulse oximetry (SpO₂) signals, body temperature, or spirometry signals.

4. The non-transitory computer program product of claim 1, wherein the signal strength is the amplitude of electric current, electric impedance, pressure, flow rate, temperature, vibration, breath rate, weight, pulse amplitude, pulse wave velocity, or frequency of physiological events within the time period.

5. The non-transitory computer program product of claim 1, wherein the accumulated signal strength in each of the visual elements is indicated by different colors, grayscales, dot densities, contour lines, or screentones.

6. The non-transitory computer program product of claim 5, further comprising one or more contrast lines surrounding the visual elements of one or more of the accumulated signal strengths.

7. A non-transitory computer program product embodied in a computer-readable medium which, when executed by one or more analysis modules, provides a visual output for presenting physiological signals, comprising:
a first axis being a logarithmic scale of frequency modulation (FM);
a second axis being a logarithmic scale of amplitude modulation (AM); and
a plurality of visual elements, each of the visual elements being defined by the first axis and the second axis, and each of the visual elements comprising a probability for quantifying statistical significance between at least two other visual outputs,
wherein each of the other visual outputs comprises a plurality of analyzed data units, and each of the analyzed data units comprises a first coordinate, a second coordinate, and a signal strength value, the first coordinate is an argument of a FM function from a transformation on a primary intrinsic mode function (IMF), the second coordinate is an argument of an AM function from a transformation on a secondary IMF, each of the primary IMF is generated from an empirical mode decomposition (EMD) of a plurality of the physiological signals, and each of the secondary IMF is generated from an EMD of the primary IMF, and the accumulated signal strength is an integral of the signal strength value of each of the analyzed data units.

8. The non-transitory computer program product of claim 7, wherein the probability for quantifying the statistical significance is a P-value.

9. A non-transitory computer program product embodied in a computer-readable medium which, when executed by one or more analysis modules, provides a visual output for presenting physiological signals, comprising:
a first axis being a logarithmic scale of frequency modulation (FM);
a second axis being a logarithmic scale of amplitude modulation (AM); and
a plurality of visual elements, each of the visual elements being defined by the first axis and the second axis, and each of the visual elements comprising an area-under-curve (AUC) between at least two other visual outputs,
wherein each of the other visual outputs comprises a plurality of analyzed data units, and each of the analyzed data units comprises a first coordinate, a second coordinate, and a signal strength value, the first coordinate is an argument of a FM function from a transformation on a primary intrinsic mode function (IMF), the second coordinate is an argument of an AM function from a transformation on a secondary IMF, each of the primary IMF is generated from an empirical mode decomposition (EMD) of a plurality of the physiological signals, and each of the secondary IMF is generated from an EMD of the primary IMF, and the accumulated signal strength is an integral of the signal strength value of each of the analyzed data units.

10. A system for analyzing physiological signals, comprising:
a detection module for detecting the physiological signals;
a transmission module for receiving the physiological signals from the detection module and delivering the physiological signals to the analysis module;
an analysis module for generating a plurality of analyzed data sets from the physiological signals, and
a visual output module for rendering a visual output space according to the analyzed data sets generated by the analysis module, and displaying a visual output,
wherein the visual output comprises a first axis representing frequency modulation (FM), a second axis representing amplitude modulation (AM), and a plurality of visual elements defined by the first axis and the second axis, and each of the visual elements comprises an accumulated signal strength and the analyzed data sets, and each of the analyzed data sets comprises a plurality of analyzed data units in a time period, each of the analyzed data units comprises a first coordinate, a second coordinate, and a signal strength value, the first coordinate is an argument of a FM function and the second coordinate is another argument of a AM function, and the accumulated signal strength is an integral of the signal strength value of each of the analyzed data units.

11. The system of claim 10, wherein the first axis is a logarithmic scale of frequency modulation (FM), the second axis is a logarithmic scale of amplitude modulation (AM), the first coordinate is a logarithmic value of the argument of the FM function, and the second coordinate is a logarithmic value of the argument of the AM function.

12. The system of claim 10, further comprising a non-transitory computer program product for presenting physiological signals, wherein the non-transitory computer program product comprises sets of instructions that, when executed by the analysis module, causes the analysis module to perform actions comprising:
1) performing empirical model decomposition (EMD) on the physiological signals to generate a set of primary intrinsic mode functions (IMF's);
2) performing the EMD on the set of primary IMFs to generate a set of secondary IMF's;
3) performing transformations on the set of primary IMF s to generate FM functions;
4) performing transformations on the set of secondary IMFs to generate AM functions; and
5) combining the AM functions and the FM functions to generate a plurality of the analyzed data sets.

13. The system of claim 10, wherein the physiological signals are electrocardiogram (EKG) signals, electromyogram (EMG) signals, electroretinography (ERG) signals, blood pressure, pulse oximetry (SpO₂) signals, body temperature, or spirometry signals.

14. The system of claim 10, wherein the signal strength is the amplitude of electric current, electric impedance, pressure, flow rate, temperature, vibration, breath rate, weight, pulse amplitude, pulse wave velocity, or frequency of physiological events within the time period.

15. The system of claim 10, wherein the accumulated signal strength in each of the visual elements is indicated by different colors, grayscales, dot densities, contour lines, or screentones.

16. The system of claim 15, further comprising one or more contrast lines surrounding the visual elements of one or more of the accumulated signal strengths.

17. A system for analyzing physiological signals, comprising:
an analysis module for generating a set of probabilities for quantifying statistical significance between at least two other visual outputs, and each of the other visual outputs comprising a plurality of analyzed data units, and each of the analyzed data units comprising a first coordinate, a second coordinate, and a signal strength value, the first coordinate being an argument of a FM function from a transformation on a primary intrinsic mode function (IMF), the second coordinate being an argument of an AM function from a transformation on a secondary IMF, each of the primary IMF being generated from an empirical mode decomposition (EMD) of a plurality of the physiological signals, and each of the secondary IMF being generated from an EMD of the primary IMF, and the accumulated signal strength being an integral of the signal strength value of each of the analyzed data units; and
a visual output module for rendering a visual output space according to the set of probabilities, and displaying a visual output,
wherein the visual output comprises a first axis of a logarithmic scale of frequency modulation (FM), a second axis of a logarithmic scale of amplitude modulation (AM), and a plurality of visual elements defined by the first axis and the second axis, and each of the visual element comprises a probability for quantifying statistical significance

18. The system of claim 17, wherein the probability for quantifying statistical significance is a P-value.

19. A system for analyzing physiological signals, comprising:
an analysis module for generating a set of area-under-curves (AUCs) between at least two other visual elements, and each of the other visual outputs comprising a plurality of analyzed data units, and each of the analyzed data units comprising a first coordinate, a second coordinate, and a signal strength value, the first coordinate being an argument of a FM function from a transformation on a primary intrinsic mode function (IMF), the second coordinate being an argument of an AM function from a transformation on a secondary IMF, each of the primary IMF being generated from an empirical mode decomposition (EMD) of a plurality of the physiological signals, and each of the secondary IMF being generated from an EMD of the primary IMF, and the accumulated signal strength being an integral of the signal strength value of each of the analyzed data units; and
a visual output module for rendering a visual output space according to the set of AUCs, and displaying an AUC visual output,
wherein the AUC visual output comprises a first axis of a logarithmic scale of frequency modulation (FM), a second axis of logarithmic scale of amplitude modulation (AM), and a plurality of AUC visual elements defined by the first axis and the second axis, and each of the AUC visual element comprises an AUC.

20. A non-transitory computer program product embodied in a computer-readable medium which, when executed by one or more analysis modules, provides a visual output for presenting physiological signals, comprising:
a first axis representing variations of signal strength of the physiological signals within a time period;
a second axis representing signal strengths of the physiological signals;
wherein zero is on a midpoint of the first axis and a threshold value is on a midpoint of the second axis, and the visual output is divided into four quadrants by the threshold value on the second axis and zero on the first axis.

21. The non-transitory computer program product of claim 20, wherein the physiological signals are transformed into one or more intrinsic mode functions (IMFs) by empirical mode decomposition (EMD), the first axis is a scale of arguments of the variations of the IMFs, and the second axis is the signal strength of the IMFs.

22. The non-transitory computer program product of claim 21, wherein the IMFs are logarithmized, the first axis is a logarithmic scale of the arguments of the variations of the IMFs, the second axis is a logarithmic scale of the signal strength of the IMFs, and a logarithmic value of the threshold value is on a midpoint of the first axis.

23. The non-transitory computer program product of claim 20, wherein the physiological signals are electrocardiogram (EKG) signals, electromyogram (EMG) signals, electroretinography (ERG) signals, blood pressure, pulse oximetry (SpO₂) signals, body temperature, or spirometry signals.

24. The non-transitory computer program product of claim 20, wherein the signal strength is an amplitude of electric current, electric impedance, pressure, flow rate, temperature, vibration, breath rate, weight, pulse amplitude, pulse wave velocity, or frequency of physiological events within a time period.

25. A system for analyzing physiological signals, comprising:
a detection module for detecting the physiological signals;
a transmission module for receiving the physiological signals from the detection module and delivering the physiological signals to the analysis module;
an analysis module for generating a primary analyzed data set;
a non-transitory computer program product for presenting physiological signals, wherein the non-transitory computer program product comprises sets of instructions that, when executed by the analysis module, causes the analysis module to perform actions comprising:
1) calculating variations of signal strengths of the physiological signals in a time period; and
2) combining the variations of the signal strengths and the signal strengths of the physiological signals to generate a primary analyzed data set;
a visual output module for rendering a visual output space according to the primary analyzed data set from the analysis module, and displaying a visual output comprising a first axis representing the variations of the signal strengths and a second axis representing the signal strengths,
wherein zero is on a midpoint of the first axis and a threshold value is on a midpoint of the second axis, and the visual output is divided into four quadrants by the threshold value on the second axis and zero on the first axis.

26. The system of claim 25, wherein the actions performed by the analysis module further comprises:
3) performing empirical mode decompositions (EMD) on the physiological signals to generate one or more intrinsic mode functions (IMFs);
4) calculating variations of the IMFs in the time period; and
5) combining the variations of the IMFs and the IMFs to generate a plurality of secondary analyzed data sets.

27. The system of claim 26, the visual output module further comprising rendering another visual output space according to the secondary analyzed data sets from the analysis module, and displaying another visual output comprising a first axis representing a scale of arguments of the variations of the intrinsic mode functions (IMFs), a second axis representing a signal strength of the IMFs,
wherein zero is on the midpoint of the first axis and another threshold value is on the midpoint of the second axis, and the another visual output is divided into four quadrants by the another threshold value on the second axis and zero on the first axis.

28. The system of claim 27, wherein the intrinsic functions (IMFs) are logarithmized, the first axis is a logarithmic scale of arguments of the variations of the IMFs, the second axis is a logarithmic scale of the signal strength of the IMFs, and a logarithmic value of the another threshold value is on the midpoint of the first axis.

29. The system of claim 25, wherein the physiological signals are electrocardiogram (EKG) signals, electromyogram (EMG) signals, electroretinography (ERG) signals, blood pressure, pulse oximetry (SpO₂) signals, body temperature, or spirometry signals.

30. The system of claim 25, wherein the signal strength is an amplitude of electric current, electric impedance, pressure, flow rate, temperature, vibration, breath rate, weight, pulse amplitude, pulse wave velocity, or frequency of physiological events within a time period.
